(19) United States
(12) Patent Application Publication
(10) Pub. No.: US 2009/0005624 A1
(43) Pub. Date: Jan. 1, 2009

(54) Integrated Processing of Methanol to Olefins

(76) Inventor: Andrea G. Bozzano, Northbrook, IL (US)

Correspondence Address: HONEYWELL INTELLECTUAL PROPERTY INC;PATENT SERVICES, 101 COLUMBIA DRIVE, P O BOX 2245 MAIL STOP AB/2B, MORRISTOWN, NJ, 07962, US

(21) Appl. No.: 12/129,020

(22) Filed: May 29, 2008

Related U.S. Application Data
(60) Provisional application No. 60/946,433, filed on Jun. 27, 2007.

(51) Int. Cl. C07C 1/20

(52) U.S. Cl. 585/639

(57) ABSTRACT

Processing schemes and arrangements for the production of olefins and, more particularly, for the production of light olefins from a methanol feedstock are provided. Such processing schemes and arrangements integrate oxygenate conversion at higher pressures and with subsequent heavy olefins conversion processing to produce additional light olefin products. In particular, this invention provides an efficient method for removal of heavy oxygenate materials such as aldehydes and ketones through the recirculation of a mixed water/methanol solvent from a reactor in which methanol is converted into dimethyl ether and water.

### CROSS-REFERENCE TO RELATED APPLICATION

This application claims priority from Provisional Application No. 60/946,433 filed Jun. 27, 2007, the contents of which are hereby incorporated by reference.

### BACKGROUND OF THE INVENTION

This invention relates generally to the conversion of oxygenates to olefins and, more particularly, to light olefins, via integrated processing.

A major portion of the worldwide petrochemical industry is involved with the production of light olefin materials and their subsequent use in the production of numerous important chemical products. Such production and use of light olefin materials may involve various well-known chemical reactions including, for example, polymerization, oligomerization, and alkylation reactions. Light olefins generally include ethylene, propylene and mixtures thereof. These light olefins are essential building blocks used in the modern petrochemical and chemical industries. A major source for light olefins in present day refining is the stream cracking of petroleum feeds. For various reasons including geographical, economic, political and diminished supply considerations, the art has long sought sources other than petroleum for the massive quantities of raw materials that are needed to supply the demand for these light olefin materials.

The search for alternative materials for light olefin production has led to the use of oxygenates such as alcohols and, more particularly, to the use of methanol, ethanol, and higher alcohols or their derivatives or other oxygenates such as dimethyl ether, diethyl ether, etc., for example. Molecular sieves such as microporous crystalline zeolite and non-zeolitic catalysts, particularly silicoaluminophosphates (SAPO), are known to promote the conversion of oxygenates to hydrocarbon mixtures, particularly hydrocarbon mixtures composed largely of light olefins.

Such processing, wherein the oxygenate-containing feed is primarily methanol or a methanol-water combination (including crude methanol), typically results in the release of significant quantities of water upon the sought conversion of such feeds to light olefins. For example, such processing normally involves the release of about 2 mols of water per mol of ethylene formed and the release of about 3 mols of water per mol of propylene formed. The presence of such increased relative amounts of water can significantly increase the potential for hydrothermal damage to the oxygenate conversion catalyst. Moreover, the presence of such increased relative amounts of water significantly increases the volumetric flow rate of the reactor effluent, resulting in the need for larger sized vessels and associated processing and operating equipment.

U.S. Pat. No. 5,714,662 to Vora et al., the disclosure of which is hereby incorporated by reference in its entirety, discloses a process for the production of light olefins from a hydrocarbon gas stream by a combination of reforming, oxygenate production, and oxygenate conversion wherein a crude methanol stream (produced in the production of oxygenates and comprising methanol, light ends, and heavier alcohols) is passed directly to an oxygenate conversion zone for the production of light olefins.

While such processing has proven to be effective for olefin production, further improvements have been desired and sought. For example, there is an ongoing desire and need for reducing the size and consequently the cost of required reaction vessels. Further, there is an ongoing desire and need for processing schemes and arrangements that can more readily handle and manage either or both the heat of reaction and byproduct water associated with such processing. Still further, there is an ongoing desire and need for processing schemes and arrangements that produce or result in increased relative amounts of light olefins.

A further issue to deal with is that the products of the oxygenate conversion zone include a C₄⁺ olefin stream. This stream or fractions of this stream can be fed to an olefin conversion process, such as an olefin cracking process or a metathesis process to improve the yield of ethylene and propylene. However, this C₄⁺ stream also contains heavy oxygenate materials, such as ketones and aldehydes which must be removed prior to further processing. One method to remove such heavy oxygenate materials was previously found to be extraction with the appropriate liquid, such as a methanol/water mixture that is a solvent for the oxygenate materials.

The present invention includes a DME reactor to first convert much of the methanol to DME and water. The reaction is not complete with some of the methanol remaining after this first conversion step. Prior to the feeding of the DME to the oxygenate conversion reactor, a separation step is required to remove the remaining methanol and much of the water normally through fractionation.

The removal of the heavy oxygenate materials and the treatment of the DME have now been found to be advantageously combined through a process that uses the methanol/water removed from the DME to in addition remove the heavy oxygenate materials such as ketones and aldehydes from the stream produced from the oxygenate conversion reaction.

### SUMMARY OF THE INVENTION

The present invention provides improved processing schemes and arrangements for the production of olefins, particularly light olefins.

The general object of the invention can be attained, at least in part, through specified methods for producing light olefins. In accordance with one embodiment, there is provided a method for producing light olefins that involves contacting a methanol-containing feedstock in a methanol conversion reactor zone with a catalyst and at reaction conditions effective to produce a methanol conversion reactor zone effluent comprising dimethyl ether and water. At least a portion of the water is removed from the methanol conversion reactor zone effluent to form a first process stream comprising dimethyl ether and having a reduced water content. A feed comprising at least a portion of the first process stream is contacted in an oxygenate conversion reactor zone with an oxygenate conversion catalyst at oxygenate conversion reaction conditions, including an oxygenate conversion reaction pressure of at least about 240 kPa absolute, effective to convert at least a portion of the feed to an oxygenate conversion product stream comprising light olefins and heavy olefins. At least a portion of the oxygenate conversion product stream heavy olefins are reacted in a heavy olefins conversion zone to form a heavy olefins conversion zone effluent stream comprising additional light olefins. At least a portion of the additional light olefins are subsequently recovered from the heavy olefins conversion zone effluent stream. At least a liquid portion of the oxygenate conversion product stream is contacted in an absorber with a solvent mixture comprising at least methanol and water. The solvent mixture is effective to absorb a significant portion of the oxygenates from the contacted portion of the oxygenate conversion product stream. At least a portion of the oxygenates absorbed from the contacted portion of the oxygenate conversion product stream is fed to the oxygenate conversion reactor for contact with the oxygenate conversion catalyst and at reaction conditions effective to convert at least a portion of the oxygenates to oxygenate conversion products.

The prior art generally fails to processing schemes and arrangements for the production of olefins and, more particularly, for the production of light olefins from an oxygenate-containing feed and which processing schemes and arrangements are as simple, effective and/or efficient as may be desired. More particularly, the prior art generally fails to provide such processing schemes and arrangements that address issues such as relating to water co-production, light olefin production with desirably increased propylene to ethylene ratios and carbon efficiency for light olefin production as simply, effectively and/or efficiently as may be desired.

A method for producing light olefins, in accordance with another embodiment, involves contacting a methanol-containing feedstock in a methanol conversion reactor zone with a catalyst and at reaction conditions effective to produce a methanol conversion reactor zone effluent comprising dimethyl ether and water. At least a portion of the water is removed from the methanol conversion reactor zone effluent to form a first process stream comprising dimethyl ether and having a reduced water content an a second process stream comprising methanol and having an increased water content compared to said first process stream. A feed comprising at least a portion of the first process stream can then be contacted in an oxygenate conversion reactor zone with an oxygenate conversion catalyst at oxygenate conversion reaction conditions effective to convert at least a portion of the feed to an oxygenate conversion product stream comprising light olefins and heavy olefins. A portion or all of the second process stream is sent to a wash column. The oxygenate conversion reaction conditions desirably include an oxygenate conversion reaction pressure in a range of at least 300 kPa absolute to 450 kPa absolute. At least a portion of the oxygenate conversion product stream heavy olefins can subsequently be reacted in a heavy olefins conversion zone via at least one of an olefin cracking reaction and a metathesis reaction to form a heavy olefins conversion zone effluent stream comprising additional light olefins. At least a portion of the additional light olefins can subsequently be recovered from the heavy olefins conversion zone effluent stream. The oxygenate conversion product stream is contacted with the second process stream so that it is washed to produce a washed olefins stream to be sent for further reaction and a waste stream comprising oxygenates and water.

There is also provided a system for producing light olefins. In accordance with one preferred embodiment, such a system includes a methanol conversion reactor zone for contacting a methanol-containing feedstock with a catalyst and at reaction conditions effective to produce a methanol conversion reactor zone effluent comprising dimethyl ether and water. A first separator is provided. The first separator is effective to separate at least a portion of the water from the methanol conversion reactor zone effluent to form a first process stream comprising dimethyl ether and having a reduced water content. An oxygenate conversion reactor zone is provided for contacting a feed comprising at least a portion of the first process stream dimethyl ether with an oxygenate conversion with a catalyst and at reaction conditions effective to convert at least a portion of the feed to an oxygenate conversion product stream comprising light olefins and heavy olefins. The system also includes a heavy olefins conversion zone effective to convert oxygenate conversion product stream heavy olefins to form a heavy olefins conversion zone effluent stream comprising additional light olefins. The system further includes a recovery zone for recovering at least a portion of the additional light olefins from the heavy olefins conversion zone effluent stream.

As used herein, references to “light olefins” are to be understood to generally refer to C₂ and C₃ olefins, i.e., ethylene and propylene.

In the subject context, the term “heavy olefins” generally refers to C₄ to C₆ olefins.

“Oxygenates” are hydrocarbons that contain one or more oxygen atoms. Typical oxygenates include alcohols and ethers, for example.

“Carbon oxide” refers to carbon dioxide and/or carbon monoxide.

References to “Cₓ hydrocarbon” are to be understood to refer to hydrocarbon molecules having the number of carbon atoms represented by the subscript “x”. Similarly, the term “Cₓ-containing stream” refers to a stream that contains Cₓ hydrocarbon. The term “Cₓ⁺ hydrocarbons” refers to hydrocarbon molecules having the number of carbon atoms represented by the subscript “x” or greater. For example, “C₄⁺ hydrocarbons” include C₄, C₅ and higher carbon number hydrocarbons. The term “Cₓ-hydrocarbons” refers to hydrocarbon molecules having the number of carbon atoms represented by the subscript “x” or less. For example, “C₄⁻ hydrocarbons” include C₄, C₃ and lower carbon number hydrocarbons.

“RWD” column or zone refers to a Reaction With Distillation column or zone such as can generally serve to combine reaction and distillation processing in a single processing apparatus.

Other objects and advantages will be apparent to those skilled in the art from the following detailed description taken in conjunction with the appended claims and drawing.

### BRIEF DESCRIPTION OF THE DRAWING

FIG. 1 is a diagram of an integrated system for the processing of an oxygenate-containing feedstock to olefins, particularly light olefins, in accordance with the invention.

FIG. 2 is a diagram of a portion of the wash column from FIG. 1 with an additional water stream entering the wash column.

Those skilled in the art and guided by the teachings herein provided will recognize and appreciate that the illustrated system or process flow diagrams have been simplified by the elimination of various usual or customary pieces of process equipment including some heat exchangers, process control systems, pumps, fractionation systems, and the like. It may also be discerned that the process flow depicted in the figures may be modified in many aspects without departing from the basic overall concept of the invention.

### DETAILED DESCRIPTION OF THE INVENTION

Oxygenate-containing feedstock can be converted to light olefins in a catalytic reaction and heavier hydrocarbons (e.g., C₄⁺ hydrocarbons) formed during such processing can be subsequently further processed to increase the light olefins (e.g., C₂ and C₃ olefins) produced or resulting therefrom. In accordance with the invention, a methanol-containing feedstock is converted to form a first process stream, that comprises dimethyl ether (DME) and having a reduced water content, which in turn is reacted to form a product mixture including light olefins and heavy olefins, with at least a portion of the heavy olefins being subsequently converted to form additional light olefin products. In the conversion of the feedstock to DME, a substantial volume of water is produced that is separated from the DME in addition to methanol that has not been converted to DME. This methanol/water stream which is a second process stream, preferably containing about 30-50% methanol, is used as the solvent for the removal of oxygenates that are among the impurities produced in the conversion of the oxygenate-containing feedstock to light olefins. As described in greater detail below, prior to such heavier hydrocarbon cracking processing, the process stream can desirably be processed by contacting at least a portion of the oxygenate conversion product stream in an absorber with a solvent mixture including at least methanol and water, as such a solvent mixture has been found to be particularly effective in the liquid-liquid absorption or liquid-liquid contact and removal of a significant portion of the oxygenates from the contacted portion of the oxygenate conversion product stream without detrimentally also absorbing significant quantities of olefins also present in the product stream.

At least a portion of the oxygenates absorbed from the contacted portion of the oxygenate conversion product stream can subsequently be processed such as via the oxygenate conversion reactor to form additional oxygenate conversion products.

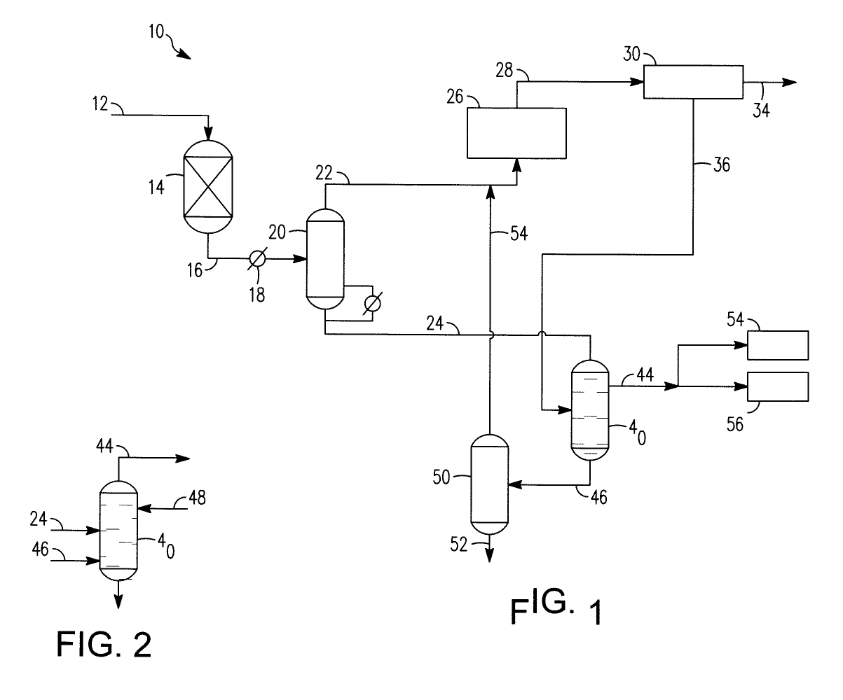

FIG. 1 schematically illustrates an integrated system, generally designated by the reference numeral 10, for processing of an oxygenate-containing feedstock to olefins, particularly light olefins, in accordance with one embodiment.

More particularly, a methanol-containing feedstock is introduced via a line 12 into a methanol conversion reactor zone 14 wherein the methanol-containing feedstock contacts with a methanol conversion catalyst and at reaction conditions effective to convert the methanol-containing feedstock to produce a methanol conversion reactor zone effluent stream comprising dimethyl ether and water, in a manner as is known in the art.

As will be appreciated by those skilled in the art and guided by the teachings herein provided, such a feedstock may be commercial grade methanol, crude methanol or any combination thereof. Crude methanol may be an unrefined product from a methanol synthesis unit. Those skilled in that art and guided by the teachings herein provided will understand and appreciate that in the interest of factors such as improved catalyst stability, embodiments utilizing higher purity methanol feeds may be preferred. Thus, suitable feeds may comprise methanol or a methanol and water blend, with possible such feeds having a methanol content of between about 65% and about 100% by weight, preferably a methanol content of between about 80% and about 100% by weight and, in accordance one preferred embodiment, a methanol content of between about 95% and about 100% by weight.

While the process conditions for such methanol conversion to dimethyl ether can vary, in practice such vapor phase process reaction can typically desirably occur at a temperature in the range of about 200° to about 300° C. (with a temperature of about 240° to about 260° C., e.g., at about 250° C., being preferred); a pressure in the range of about 200 to about 1500 kPa (with a pressure in the range of about 400 to about 700 kPa, e.g., at about 500 kPa, being preferred); and a weight hourly space velocity (“WHSV”) in the range of about 2 to about 15 hr⁻¹, with a WHSV in the range of about 3 to about 7 hr⁻¹, e.g., about 5 hr⁻¹, being preferred). In practice, a rate of conversion of methanol to dimethyl ether of about 80 percent or more is preferred.

The methanol conversion reactor zone effluent stream is introduced via a line 16 into a separator section 20 such as composed of one or more separation units such as known in the art wherein at least a portion of the water is removed therefrom to form a first process stream comprising dimethyl ether and having a reduced water content in a line 22 and a stream composed primarily of water in combination with unreacted methanol in line 24. A cooler device 18 may be appropriately disposed prior to the separator section 20 such as to facilitate desired water separation.

For example, such water separation can desirably be carried out in a flash drum or, if a more complete separation is desired, in a distillation column separation unit. In practice, it is generally desirable to remove at least about 75 percent or more, preferably at least about 90 percent or more of the produced water.

Those skilled in the art and guided by the teachings herein provided will appreciate that remaining unreacted methanol can either partition in a separation unit overhead stream or a separation unit bottoms stream or both, for further processing as herein described. For example, methanol in such separation unit bottoms stream can, if desired, be recovered (such as through or by a stripper column) and recycled to the methanol conversion reactor zone 14.

The first process stream or at least a portion thereof, is fed or introduced via the line 22 into an oxygenate conversion reactor section 26 wherein the feed contacts with an oxygenate conversion catalyst at reaction conditions effective to convert at least a portion of the feed to an oxygenate conversion product stream comprising fuel gas hydrocarbons, light olefins, and C₄⁺ hydrocarbons, including a quantity of heavy hydrocarbons, in a manner as is known in the art, such as, for example, utilizing a fluidized bed reactor.

Reaction conditions for the conversion of oxygenates such as dimethyl ether, methanol and combinations thereof, for example, to light olefins are known to those skilled in the art. Preferably, in accordance with particular embodiments, reaction conditions comprise a temperature between about 200° and about 700° C., more preferably between about 300° and 600° C., and most preferably between about 400° and about 550° C. As will be appreciated by those skilled in the art and guided by the teachings herein provided, the reactions conditions are generally variable such as dependent on the desired products. The light olefins produced can have a ratio of ethylene to propylene of between about 0.5 and about 2.0 and preferably between about 0.75 and about 1.25. If a higher ratio of ethylene to propylene is desired, then the reaction temperature is higher than if a lower ratio of ethylene to propylene is desired. The preferred feed temperature range is between about 80° and about 210° C. More preferably the feed temperature range is between about 110° and 210° C. In accordance with one preferred embodiment, the temperature is desirably maintained below 210° C. to avoid or minimize thermal decomposition.

In accordance with certain preferred embodiments, it is particularly advantageous to employ oxygenate conversion reaction conditions including an oxygenate conversion reaction pressure of at least 240 kPa absolute. In certain preferred embodiments, an oxygenate conversion reaction pressure in a range of at least 240 kPa absolute to 580 kPa absolute is preferred. Moreover, in certain preferred embodiments an oxygenate conversion reaction pressure of at least 300 kPa absolute and such as in a range of at least 300 kPa absolute to 450 kPa absolute may be preferred. Those skilled in the art and guided by the teachings herein provided will appreciate that through such operation at pressures higher than normally utilized in conventional oxygenate-to-olefin, particularly methanol-to-olefin (e.g., “MTO”) processing, significant reductions in reactor size (e.g., reductions in size of the oxygenate conversion reactor can be realized). For example, in view of the ratio of pressure between normal operation and higher pressure operation in accordance herewith, reductions in reactor size of at least about 20 percent or more, such as reductions in reactor size of about 33 percent or more can be realized through such higher pressure operation.

In practice, oxygenate conversions of at least about 90 percent, preferably of at least about 95 percent and, in at least certain preferred embodiments, conversions of 98 to 99 percent or more can be realized in such oxygenate-to-olefin conversion processing.

The oxygenate conversion reactor section 26 produces or results in an oxygenate conversion product or effluent stream generally comprising fuel gas hydrocarbons, light olefins, heavy olefins and other C₄⁺ hydrocarbons as well as by-product water in a line 28. The oxygenate conversion effluent stream or at least a portion thereof is appropriately processed such as through a fractionation section 30 such as to form a resulting compressed oxygenate conversion product stream in a line 34 and a C₄⁺ olefin and other waste product, such as oxygenated by-products such as low molecular weight aldehydes and organic acids in a line 36.

FIG. 1 has been simplified to show a product stream line 34 such as generally composed of at least one and usually more end product materials and a process stream line 36 such as sent for further processing in accordance with the invention as more fully described below. As described in greater detail below and in accordance with one preferred embodiment (see FIG. 2, for example), such a treatment and hydrocarbon recovery zone may desirably include one or more unit operations whereby the oxygenate conversion product stream can be treated, such as via a liquid-liquid absorption, extraction or contact and removal with a methanol and water solvent mixture to remove and desirably recover selected species, such as oxygenates, such as DME.

Stream 24 comprising a mixture of water and methanol is sent to a wash column 40 to remove oxygenates from the stream in line 36. A purified C₄⁺ stream of olefins (also referred to as a recirculate stream) is then sent for further processing in line 44 in an olefin cracking reactor (not shown) or metathesis reaction zone (not shown) or other reactor. The waste stream 46 largely comprising water, methanol and other oxygenates is then sent to an oxygenate stripper 50 in which waste water 52 is removed for recycle or other use and stream 54 comprising methanol and other oxygenates is returned to line 22 for passage to oxygenate conversion reactor 26.

The bottoms stream from the fractionation is therefore routed to a liquid extraction column for contacting with oxygenate containing heavy olefins. In this column, the solvent extracts oxygenates as well as minor amounts of olefins. The solvent exiting the column bottom is then routed to a stripper column, in which the methanol and other oxygenates are stripped out. From here, they can be routed directly to the MTO reactor for conversion. Any extracted olefins and other oxygenates will go overhead with the methanol to the MTO reactor.

In some instances, as shown in the attached diagram, the wash column will have two sections, an upper water wash section and a lower methanol/water wash section. The purpose of the upper section is to remove any residual methanol from the hydrocarbons exiting the column. The upper section further serves the purpose of adjusting the water/methanol concentration in the lower section. If the methanol concentration in the lower section is too high, there is the danger of removing a substantial amount of the olefin to the column bottoms. This is not advantageous since it recycles these heavy olefins to the MTO. Hence, by increasing the water to the upper section, further dilution of the methanol can be achieved. The upper section can also contains water draw off, in case no net water to the bottom section is desired.

Those skilled in the art and guided by the teachings herein provided will appreciate that the system integration of the methanol conversion reactor zone whereby methanol can desirably be converted to dimethyl ether, with the subsequent removal of byproduct water reduces the volumetric flow through the reactor and hence reduces the size of the reactor. Moreover, such removal of water can advantageously reduce the hydrothermal severity of the reactor. Still further, the system integration of such a methanol conversion reactor zone can desirably result in removal of a significant portion of the heat of reaction such as to allow operation with reduced cooling requirements (e.g., operation with the removal of one or more catalyst coolers from the reactor). Yet still further, possible processing disadvantages such as due to possible increased selectivity to heavy hydrocarbons, particularly heavy olefins, are desirably minimized or avoided through the system integration of appropriate heavy olefins conversion zone as herein described.

Those skilled in the art and guided by the teachings herein provided will additionally note that the use of DME as feed to an oxygenate-to-olefins conversion reactor unit can present operational advantages over the use of other oxygenate feed materials, such as during the startup of the oxygenates-to-olefins reactor. For example, due to its relatively low boiling point, DME can be introduced as a gas into a cold reactor without the possibility of condensation, and can be used as a heating medium to increase the reactor temperature. In contrast, higher boiling oxygenate feedstock materials such as methanol, ethanol, etc, may require the reactor to be preheated such as by or through some other heating medium to avoid condensation in the reactor. Those skilled in the art will recognize and appreciate the importance of avoiding gas condensation in a fluidized bed system, and will recognize the advantages of a simplified startup procedure using DME as a feed material in such processing.

To further the understanding of the subject development, reference is now made to FIG. 2. FIG. 2 illustrates an additional water line 48 that is introduced for stripping out residual methanol from the C₄⁺ olefins system and to adjust the ratio of water to methanol within the wash column. As in FIG. 1, FIG. 2 shows the wash column 40, methanol/water feed 24, C₄⁺ olefin feed 46 and line 44 in which the C₄⁺ olefins that have been treated are removed.

The C₄⁺ hydrocarbon stream or a selected portion thereof in the line 44 is introduced into an olefin cracking reactor 54 in which additional C₂ and C₃ product is produced to be added to the product stream 34.

The C₄⁺ hydrocarbon stream or a selected portion thereof in the line 44 can alternatively be introduced into a heavy olefins conversion zone 56 in the form of a metathesis reaction section and under effective conditions to produce a metathesis effluent comprising propylene.

The metathesis reaction can generally be carried out under conditions and employs catalysts such as are known in the art. In accordance with one preferred embodiment, a metathesis catalyst such as containing a catalytic amount of at least one of molybdenum oxide and tungsten oxide is suitable for the metathesis reaction. Conditions for the metathesis reaction generally include reaction temperature ranging from about 20° to about 450° C., preferably 250° to 350° C., and pressures varying from about atmospheric to upwards of 20.6 MPag (3000 psig), preferably between 3000 and 3500 kPag (435 to 510 psig), although higher pressures can be employed if desired. In general, the metathesis equilibrium for propylene production is generally favored by lower temperatures.

Catalysts which are active for the metathesis of olefins and which can be used in the process of this invention are of a generally known type. The disproportionation (metathesis) of butene with ethylene can, for example, be carried out in the vapor phase at about 300° to 350° C. and about 0.5 MPa absolute (75 psia) with a WHSV of 50 to 100 and a once-through conversion of about 15% or more, depending on the ethylene to butene ratio.

Such metathesis catalysts may be homogeneous or heterogeneous, with heterogeneous catalysts being preferred. The metathesis catalyst preferably comprises a catalytically effective amount of transition metal component. The preferred transition metals for use in the present invention include tungsten, molybdenum, nickel, rhenium, and mixtures thereof. The transition metal component may be present as elemental metal and/or one or more compounds of the metal. If the catalyst is heterogeneous, it is preferred that the transition metal component be associated with a support. Any suitable support material may be employed provided that it does not substantially interfere with the feedstock components or the lower olefin component conversion. Preferably, the support material is an oxide, such as silica, alumina, titania, zirconia and mixtures thereof. Silica is a particularly preferred support material. If a support material is employed, the amount of transition metal component used in combination with the support material may vary widely depending, for example, on the particular application involved and/or the transition metal being used. Preferably, the transition metal comprises about 1% to about 20%, by weight (calculated as elemental metal) of the total catalyst. The metathesis catalyst advantageously comprises a catalytically effective amount of at least one of the above-noted transition metals capable of promoting olefin metathesis. The catalyst may also contain at least one activating agent present in an amount to improve the effectiveness of the catalyst. Various activating agents may be employed, including activating agents which are well known in the art to facilitate metathesis reactions. Light olefin metathesis catalysts can, for example, desirably be complexes of tungsten (W), molybdenum (Mo), or rhenium (Re) in a heterogeneous or homogeneous phase.

As will be appreciated by those skilled in the art and guided by the teachings herein provided, such system integration of a heavy olefins conversion zone in the form of a metathesis reaction section can at least in part counteract increased selectivity to heavy hydrocarbons, e.g., heavy olefins, due to increased pressure operation.

The present invention is described in further detail in connection with the following examples which illustrate or simulate various aspects involved in the practice of the invention. It is to be understood that all changes that come within the spirit of the invention are desired to be protected and thus the invention is not to be construed as limited by these examples.

A person skilled in the art and guided by the teachings herein provided will appreciate and recognize that as a fluidized reactor system typically comprises a major cost component of an operating plant, significant reductions in reactor size and corresponding savings in reactor and catalyst inventory costs associated therewith can be realized through the practice of the invention.

The invention thus provides processing schemes and arrangements for the production of olefins and, more particularly, for the production of light olefins from an oxygenate-containing feed and which processing schemes and arrangements are advantageously simpler, more effective and/or more efficient than heretofore been generally available.

The invention illustratively disclosed herein suitably may be practiced in the absence of any element, part, step, component, or ingredient which is not specifically disclosed herein.

While in the foregoing detailed description this invention has been described in relation to certain preferred embodiments thereof, and many details have been set forth for purposes of illustration, it will be apparent to those skilled in the art that the invention is susceptible to additional embodiments and that certain of the details described herein can be varied considerably without departing from the basic principles of the invention.

1. A method for producing light olefins, said method comprising:
a) contacting a methanol-containing feedstock in a methanol conversion reactor zone with a catalyst and at reaction conditions effective to produce a methanol conversion reactor zone effluent comprising dimethyl ether and water;
b) removing at least a portion of the water from the methanol conversion reactor zone effluent to form a first process stream comprising dimethyl ether and having a reduced water content and a second process stream comprising methanol and having an increased water content compared to said first process stream;
c) sending a portion or all of said second process stream to a wash column;
d) contacting a feed comprising at least a portion of the first process stream in an oxygenate conversion reactor zone with an oxygenate conversion catalyst at oxygenate conversion reaction conditions effective to convert at least a portion of the feed to an oxygenate conversion product stream; and
e) sending said oxygenate conversion product stream to said wash column wherein said second process stream washes said oxygenate conversion product stream to produce a washed olefins stream to be sent for further reaction and a waste stream comprising oxygenates and water.

2. The method of claim 1 wherein said oxygenate conversion product stream comprises light olefins and heavy olefins.

3. The method of claim 1 wherein said oxygenate conversion product stream is separated into at least one light olefin product stream and a recirculate stream comprising C₄⁺ olefins.

4. The method of claim 1 wherein said recirculate stream is sent for further reaction.

5. The method of claim 1 wherein said waste stream is sent to a separation step to produce an oxygenate stream to return to said oxygenate conversion reactor zone.

6. The method of claim 1 wherein said second process stream comprises from about 30-50 wt-% methanol.

7. The method of claim 1 wherein a water stream is introduced to remove methanol from said heavy olefin stream and to adjust the ratio of water to methanol within said wash column.

8. The method of claim 7 wherein said water stream is introduced into said wash column to remove methanol from said heavy olefin stream and to adjust the ratio of water to methanol within said wash column.

9. The method of claim 1 wherein the reaction of at least a portion of the oxygenate conversion product stream heavy olefins comprises at least one of an olefin cracking reaction and a metathesis reaction.

10. The method of claim 9 wherein, prior to the reaction of at least a portion of the oxygenate conversion product stream heavy olefins, the method additionally comprises at least partially separating the light olefins from the heavy olefins of the oxygenate conversion product stream.

11. The method of claim 10 wherein the reaction of at least a portion of the oxygenate conversion product stream heavy olefins comprises cracking at least a portion of the separated heavy olefins to form a cracked olefin effluent comprising C₂ and C₃ olefins.

12. The method of claim 11 wherein the light olefins of the oxygenate conversion product stream comprise a quantity of C₂ olefins and the heavy olefins of the oxygenate conversion product stream comprise a quantity of C₄ olefins and wherein the reaction of at least a portion of the oxygenate conversion product stream heavy olefins comprises contacting at least a portion of the C₄ olefins with at least a portion of the C₂ olefins in a metathesis section at effective conditions to produce a metathesis effluent comprising C₃ olefins.

13. The method of claim 1 wherein the contacting of the methanol-containing feedstock in the methanol conversion reactor zone with a catalyst and at reaction conditions effective to produce a methanol conversion reactor zone effluent comprising dimethyl ether and water and the removing of at least a portion of the water from the methanol conversion reactor zone effluent to form a first process stream comprising dimethyl ether and having a reduced water content occurs concurrently in a single reaction with distillation zone.

14. A system for producing light olefins, said system comprising:
a methanol conversion reactor zone for contacting a methanol-containing feedstock with a catalyst and at reaction conditions effective to produce a methanol conversion reactor zone effluent comprising dimethyl ether and water;
a first separator effective to separate at least a portion of the water from the methanol conversion reactor zone effluent to form a first process stream comprising dimethyl ether and having a reduced water content and a second process stream comprising methanol and having a higher water content;
a passage to send a portion or all of said second process stream to a wash column;
an oxygenate conversion reactor zone for contacting a feed comprising at least a portion of the first process stream dimethyl ether with an oxygenate conversion with a catalyst and at reaction conditions effective to convert at least a portion of the feed to an oxygenate conversion product stream comprising light olefins and heavy olefins; and
a wash column wherein a stream comprising said heavy olefins and oxygenates is contacted with said second process stream to produce a heavy olefin stream and a waste stream comprising oxygenates and water.

15. The system of claim 14 additionally comprising a second separator effective to at least partially separate the light olefins from the heavy olefins of the oxygenate conversion product stream.

16. The system of claim 14 wherein the heavy olefins conversion zone comprises an olefin cracking reactor section to crack at least a portion of the separated heavy olefins to form a cracked olefin effluent comprising C₂ and C₃ olefins.

17. The system of claim 16 wherein the light olefins of the oxygenate conversion product stream comprise a quantity of C₂ olefins and the heavy olefins of the oxygenate conversion product stream comprise a quantity of C₄ olefins and wherein the heavy olefins conversion zone comprises a metathesis section wherein at least a portion of the C₄ olefins metathesize with at least a portion of the C₂ olefins to produce a metathesis effluent comprising C₃ olefins.
